(19) United States
(12) Patent Application Publication
(10) Pub. No.: US 2026/0237518 A1
(43) Pub. Date: Aug. 13, 2026

(54) AI-POWERED WEB PLATFORM FOR HEALTH ANALYTICS

(71) Applicants: Robert Fisher, Lakewood Ranch, FL (US); Richard Fessler, Lakewood Ranch, FL (US)

(72) Inventors: Robert Fisher, Lakewood Ranch, FL (US); Richard Fessler, Lakewood Ranch, FL (US)

(73) Assignee: Health Magnet Holdings, Lakewood Ranch, FL (US)

(21) Appl. No.: 19/454,588

(22) Filed: Jan. 21, 2026

Related U.S. Application Data
(60) Provisional application No. 63/757,055, filed on Feb. 11, 2025.

(51) Int. Cl. G16H 50/30; G06F 16/958; G16H 40/67

(52) U.S. Cl. CPC G16H 50/30; G06F 16/958; G16H 40/67

(57) ABSTRACT

The present disclosure relates to a system for continuous health-related data aggregation and AI-driven health analytics. The system includes a plurality of data input interfaces to collect a plurality of physiological measurements for a plurality of users. The system also includes a cloud-based server which stores and processes the plurality of physiological measurements, for creating individualized baselines for each user of the plurality of users, in addition to a modular AI engine to implement a plurality of disease-specific modules on the collected physiological measurements using the individualized baselines. Further, a unified web dashboard depicting a clinician facing interface and a consumer interface provides real-time risk indicators for clinicians and personal health insights to the plurality of users, respectively. The disclosed system offers a monitoring platform for multiple diseases with a capability to integrate newly developed or third-party disease-specific modules without affecting the present functionalities.

### FIELD OF THE INVENTION

Embodiments of the present invention relate to the field of software-based health analytics, in general and specifically relates to providing remote monitoring solutions and intervention through AI-driven modules.

### DESCRIPTION OF THE RELATED ART

Wearable devices, such as smartwatches and fitness trackers, have become widely adopted and enable continuous monitoring of physiological parameters of users. In addition, sensor-based health monitoring devices deployed in home environments facilitate the collection of physiological measurements, including step count, heart rate variability (HRV), blood pressure, sleep metrics, and stress indicators, often in real time.

Individuals affected by chronic disease conditions, such as diabetes mellitus and hypertension, who are at elevated risk of cardiovascular events including heart failure and stroke, frequently self-monitor their physiological parameters on a regular basis. However, individuals who appear to be healthy but maintain unhealthy lifestyle patterns, such as limited physical activity, may also be at increased risk and often do not consistently monitor their physiological parameters.

Physical symptoms, such as persistent fatigue, may present as isolated or nonspecific health issues, causing users to overlook their association with underlying or emerging disease conditions. Consequently, physiological signals, such as reduced activity levels, restlessness, altered heart rate variability (HRV), and disrupted sleep that may be indicative of developing health conditions are often not recognized until symptoms become acute.

By way of an example, mental health conditions, including depression and anxiety, frequently remain undetected or are identified at a late stage in individuals, which may result in severe adverse mental health outcomes.

The wearable devices and sensor-based health monitoring devices deployed in home environments generate large volumes of raw physiological data, including step count, heart rate variability (HRV), blood pressure, sleep metrics, and stress indicators. Conventional telehealth platforms associated with such devices are generally limited to transmitting these raw data sets to clinicians or users. Review and interpretation of the data typically require manual analysis by the clinicians, thereby limiting scalability and timely clinical assessment. Thus, effective conversion of raw physiological data into actionable health insights remains a significant technical challenge.

Furthermore, existing artificial intelligence (AI)-driven solutions are commonly directed toward specific disease conditions, such as congestive heart failure (CHF) monitoring, stroke risk prediction, or mental health screening. These solutions often operate independently of one another, requiring separate monitoring and evaluation. Hence, the clinicians and users are required to review outputs from multiple systems to assess overall health status, which complicates interpretation and limits comprehensive assessment.

### SUMMARY OF THE INVENTION

Embodiments of the present invention may relate to a system for continuous health-related data aggregation and AI-driven health analytics. The system may include a plurality of data input interfaces which are configured to collect a plurality of physiological measurements for a plurality of users. The system may further include a cloud-based server configured to store and process the plurality of physiological measurements, for creating individualized baselines for each user of the plurality of users. Also, the system includes a modular AI engine configured to implement a plurality of disease-specific AI modules on the collected physiological measurements using the individualized baselines. In addition, a unified web dashboard is part of the disclosed system, generated upon execution of the plurality of disease-specific AI modules. The unified web dashboard includes a clinician facing interface and a consumer interface. The clinician facing interface provides real-time risk indicators for the clinicians. The consumer interface provides personal health insights to the plurality of users. By collecting and storing, a plurality of physiological measurements for a plurality of users, a record of the plurality of users is created. The record can be analyzed to generate a personalized baseline for a particular user for comparison with future physiological measurements of that particular user and further risk analysis of disease conditions.

In accordance with an embodiment of the present invention, the physiological measurements may be collected from one of wearable devices and sensor-based health monitoring devices.

In accordance with an embodiment of the present invention, the physiological measurements may be collected from a sensor-based health monitoring device including one or more sensors configured to collect at least one physiological measurement of the user in a home environment. The disclosed invention uses the wearable devices or sensor-based health monitoring devices to collect physiological measurement(s) which facilitates in continuously tracking health signals and respond quickly when patients show early signs of disease conditions through abnormal physiological measurements.

In accordance with an embodiment of the present invention, the individualized baselines may be generated by storing and processing the physiological measurements for a pre-defined time period.

In accordance with an embodiment of the present invention, the disease-specific AI modules relate to diseases from one of congestive heart failure (CHF) risk, stroke risk, and behavioral health, etc. By monitoring different aspects of health, like vascular, behavioral, systolic, activity and sleep, the disease-specific AI modules help in identifying different diseases.

In accordance with an embodiment of the present invention, the modular AI engine may include at least one large language model (LLMs) configured to generate text-based clinical summaries of data trends of the user's physiological measurements. By providing text-based summaries, through LLM's, the process of preparing summary notes by the clinicians for symptoms or disease analysis of users is obviated. Herein, it is to be noted that the users may be referred to as patients if their physiological measurements are analyzed to be anomalous with respect to the individualized baselines.

In accordance with an embodiment of the present invention, the unified web dashboard may be communicatively connected to the cloud-based server and may be generated upon combining analysis from the implementation of the plurality of disease-specific AI modules. By aggregating the analysis, from the multiple disease-specific AI modules, the disclosed invention facilitates in providing a unified platform (the unified web dashboard) to the clinicians for comprehensive understanding of complete health of the patients.

In accordance with an embodiment of the present invention, the cloud-based server may be configured to store personal data of the plurality of the users, in compliance with healthcare privacy regulations. The storage of the personal data of the users in the cloud-based server enables fetching the user data at any point of time when required for health risk analysis thereby removing any need for searching the data manually and speeding up the detection of disease condition.

In accordance with an embodiment of the present invention, the system may include an interface to enable integration or updation of AI models, without disrupting existing services provided by the unified web dashboard. Thus, if a new disease detecting model needs to be added or an existing disease detecting model needs to be updated in the same unified web dashboard then it can be done easily because of the adaptability of the system. Additionally, the integration or addition or updation of the disease detecting AI models can be done without effecting any existing functionality and therefore the services provided by the existing AI models.

In accordance with an embodiment of the present invention, the real-time risk indicators can be displayed as risk scores, data trends, or the personal health insights. The personal health insights may be displayed as specific exercise suggestions. Thus, the risk scores and data trends enable timely clinical intervention to prevent critical health situations and, in moderate-risk scenarios, facilitate user guidance by recommending lifestyle adjustments, including modification of exercise routines.

Further, embodiments of the present invention may also include a method for performing continuous clinical surveillance using disease-specific AI modules. The method includes continuously receiving biometric data of a plurality of patients and storing the biometric data in a database. The method may also include running an AI-driven disease-specific module that calculates risk scores for targeted disease conditions based on the stored biometric data. Further, the risk scores are displayed to clinicians and patients. Upon detecting significant escalations in the risk scores in real-time, the method includes prompting interventions with the clinicians or following-up with the patients. Through the disclosed method, a clinical pathway is automated which is based on user's physiological measurements and accordingly the clinicians can continuously track health signals through the assessed health risk status and respond quickly when patients show early signs of a disease condition.

In accordance with an embodiment of the present invention, the risk scores for targeted disease conditions may be calculated by a composite scoring engine based on threshold value detection of an individualized baseline of the targeted disease conditions and trending patterns of the biometric data. The composite scoring engine may refer to an AI or ML based engine to generate a composite score (CS) that reflects the individual's health risk level. The AI or ML based engine may apply smoothing/rolling averages to the historical trends or graphs so as to avoid rapid toggling between states. Also, the composite scoring engine may parallelly process multiple types of data inputs (ex: biometric data) thereby speeding up the overall inference process of risk analysis.

In accordance with an embodiment of the present invention, the risk scores may be indicated at least in a color-coded, text-based format, and graph-based format. By way of presentation of the risk scores which serve as the critical health related information in color/picture formats or text-based format or other different ways, the clinician may easily differentiate a normal situation from an emergency situation. Consequently, the clinician may offer faster suggestions or treatment to patients. In another instance, the clinician may move on to the next patient if one case is resolved just by checking the risk status (less critical and do not need attention).

In accordance with an embodiment of the present invention, the targeted conditions may be one of stroke risk, depression, and congestive heart failure (CHF), etc.

In accordance with an embodiment of the present invention, the method includes a feedback loop through which the clinician or patient-confirmed outcomes refine the AI modules'predictive capabilities over time.

In accordance with an embodiment of the present invention, the clinicians and patients are authorized to remote therapeutic monitoring (RTM) guidelines by way of an agreement. The authorization could be done through a direct login to a website or downloading an application for the continuous clinical surveillance on a user equipment like a mobile device or a tablet.

In accordance with an embodiment of the present invention, the interventions may may include sending an alert or a short message from a telehealth consultant to one of a family member or a relevant healthcare professional and emergency services.

In accordance with an embodiment of the present invention, the follow-up may include an in-person appointment with the clinician or change in type or schedule of medications of the patient based on the risk level. Thus, if the clinician wants to look into the actual values of any of the biometric data (the biometric data includes information about a plurality of biometric parameters) for any particular person, the clinician may do so through the website or application on his/her mobile device thereby providing a quick overview of the patient's health and take action based on judging the parameters.

In accordance with an embodiment of the present invention, significant escalations in the risk scores correspond to threshold-exceeding changes, either consistent or sudden, in one or more biometric parameters pertaining to the biometric data. Herein, the changes are indicative of an elevated risk of an adverse physiological condition, like CHF decompensation or an imminent stroke, thereby triggering an alert to the clinician for urgent clinical evaluation.

In accordance with an embodiment of the present invention, the biometric data may be fetched from a plurality of wearable devices. The wearable device may include one of a smartwatch, a smart bracelet, a smart belt, an earbud, a smart ring, and a smart clothing remotely connected to the database. The disclosed method is capable of being implemented with multiple wearable devices which could be worn by the patient and are also connected to the cloud-based server for biometric data monitoring.

Additionally, embodiments of the present invention may also include a method of deploying a new disease-specific AI module in an existing AI-powered monitoring platform. The method includes providing an interface for ingesting new disease-specific AI modules. The interface may be part of a unified web dashboard, generated upon execution of a plurality of disease-specific AI modules in a virtualized execution environment. The disclosed method may also include integrating a plurality of new disease-specific AI modules within the virtualized execution environment. In addition, the method may include implementing the new disease-specific AI modules into the clinician and user-facing dashboards and notifications, without disrupting functionality of existing modules.

In accordance with an embodiment of the present invention, the plurality of disease-specific AI modules and the new disease-specific AI modules are integrated in a containerized environment. As a result of integrating the new disease-specific AI modules within the virtualized execution or containerized environment and implementing the same into the existing AI-powered monitoring platform, a seamless detection of new disease conditions is possible without any extra burden on operating capabilities of the existing AI-powered monitoring platform. Therefore, the disclosed invention is a resource efficient method of adding new capabilities to the AI-powered monitoring platform.

In accordance with an embodiment of the present invention, the interface may be a standardized application programming interface (API). Accordingly, the present method can be implemented on a standard API and do not require any specific interface for its optimal functioning.

In accordance with an embodiment of the present invention, the existing modules may include a plurality of disease-specific AI modules relating to diseases from one of congestive heart failure (CHF) risk, stroke risk, and behavioral health, etc. The existing modules may receive physiological measurements pertaining to various parameters, such as heart rate (HR), heart rate variability (HRV), blood oxygen saturation (SpO₂) , steps or activity, sleep metrics, and stress scores. Thus, a vast amount of raw information can be gathered which can facilitate in risk analysis of disease conditions.

The invention could also extend to other areas of healthcare or other fields where there is a demand for analytics, finding trends, and comprehensive insights, among others.

### BRIEF DESCRIPTION OF THE DRAWINGS

So that the manner in which the above-recited features of the present invention is understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

The invention herein will be better understood from the following description with reference to the drawings, in which:

FIG. 1 illustrates a system architecture for continuous health-related data aggregation and AI-driven health analytics, in accordance with an embodiment of the present invention;

FIG. 2 illustrates a block diagram of the system components and modules that cooperate to perform the operations of the invention, in accordance with an embodiment of the present invention;

FIG. 3 illustrates an exemplary layered system architecture for continuous health-related data aggregation and AI-driven health analytics, in accordance with one or more embodiments of the present disclosure.

FIG. 4(a) illustrates an exemplary view of a unified web dashboard depicting AI-driven health analytics, in accordance with an embodiment of the present invention;

FIG. 4(b) illustrates another exemplary view of a unified web dashboard depicting AI-driven health analytics, in accordance with an embodiment of the present invention;

FIG. 5 illustrates a flowchart of a method performing continuous clinical surveillance using disease-specific AI modules, in accordance with an embodiment of the present invention; and

FIG. 6 illustrates a flowchart of a method of deploying a new disease-specific AI module in an existing AI-powered monitoring platform, in accordance with an embodiment of the present invention.

It should be noted that the accompanying figures are intended to present illustrations of exemplary embodiments of the present disclosure. These figures are not intended to limit the scope of the present disclosure. It should also be noted that the accompanying figures are not necessarily drawn to scale.

### DETAILED DESCRIPTION OF THE INVENTION

In the following detailed description, numerous specific details are set forth in order to provide a thorough understanding of the embodiment of the invention as illustrative or exemplary embodiments of the invention, specific embodiments in which the invention may be practiced are described in sufficient detail to enable those skilled in the art to practice the disclosed embodiments. However, it will be obvious to a person skilled in the art that the embodiments of the invention may be practiced with or without these specific details. In other instances, well-known methods, procedures and components have not been described in detail so as not to unnecessarily obscure aspects of the embodiments of the invention.

The following detailed description is, therefore, not to be taken in a limiting sense, and the scope of the present invention is defined by the appended claims and equivalents thereof. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. References within the specification to “one embodiment,” “an embodiment,” “embodiments,” or “one or more embodiments” are intended to indicate that a particular feature, structure, or characteristic described in connection with the embodiment is included in at least one embodiment of the present invention.

The terminology employed in the present disclosure is utilized to delineate specific embodiments and does not aim to restrict the scope of the invention. In this context, the term “and/or” encompasses all possible combinations of one or more items listed in association. Further, the terms “a” and “an” herein do not denote a limitation of quantity, but rather denote the presence of at least one of the referenced items.

The conditional language used herein, such as, among others, “can,” “may,” “might,” “may,” “e.g.,” and the like, unless specifically stated otherwise, or otherwise understood within the context as used, is generally intended to convey that certain embodiments include, while other embodiments do not include, certain features, elements and/or steps.

Unless explicitly defined otherwise, all terms, including technical, technological, engineering, and scientific terminology, utilized in this document are presumed to carry the same connotations as commonly understood by individuals possessing ordinary skill in the pertinent field to which this invention pertains. Moreover, it is emphasized that terms, including those cataloged in commonly referenced dictionaries, should be construed to align with their intended meaning within the context of the relevant art and the disclosures provided herein. Any interpretations of terms should refrain from adopting an excessively formal or idealized stance unless explicitly delineated within the present disclosure.

When discussing the invention, it is important to recognize that various techniques and steps are disclosed, each offering distinct advantages and capable of being employed independently or in combination with one another. Therefore, this description avoids redundant enumeration of all possible combinations of individual steps to maintain clarity. However, it should be noted that such combinations are fully encompassed within the scope of the invention and the accompanying claims. Consequently, the specification and claims should be interpreted with the understanding that these combinations are permissible and fall within the ambit of the invention.

When perceiving the arrows between the components, it is understood that the direction of the arrow is only to depict the flow of the request and response highlighting the upstream and downstream systems and it does not restrict or omit the possibility of data that can flow in either direction of the arrow. When there is no arrow connecting any two or more components it only addresses an optimized path flow to achieve the desired goals and meet the needs and it does not restrict or omit the possibility of connectivity required to address any alternate flows the system needs to address for the optimal function.

As used herein, a ‘module’ which can be configured to apply trained AI models may be implemented in hardware, software, firmware, or any combination thereof, and may include rule-based logic, machine-learning logic, or hybrid approaches.

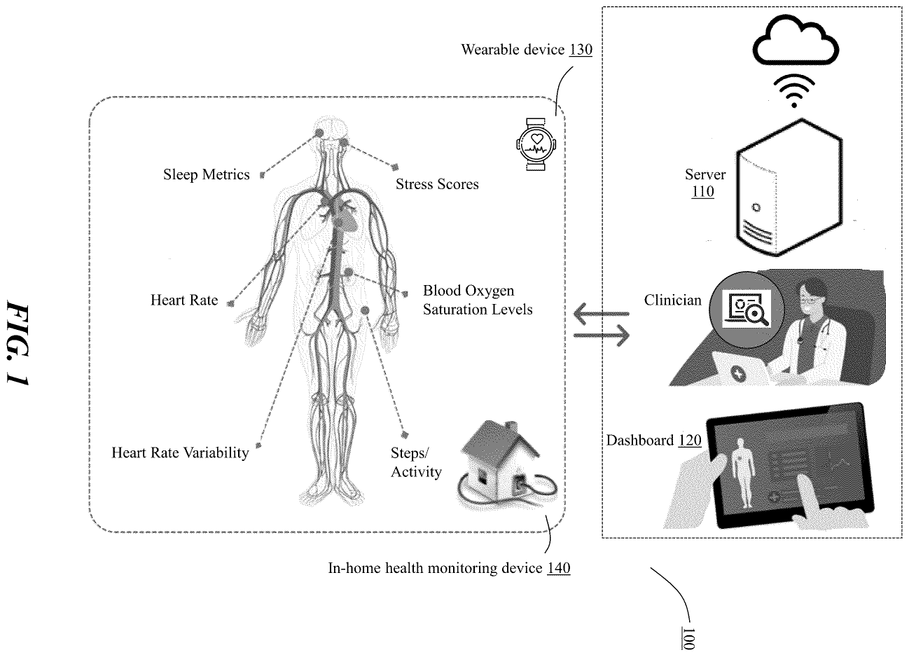

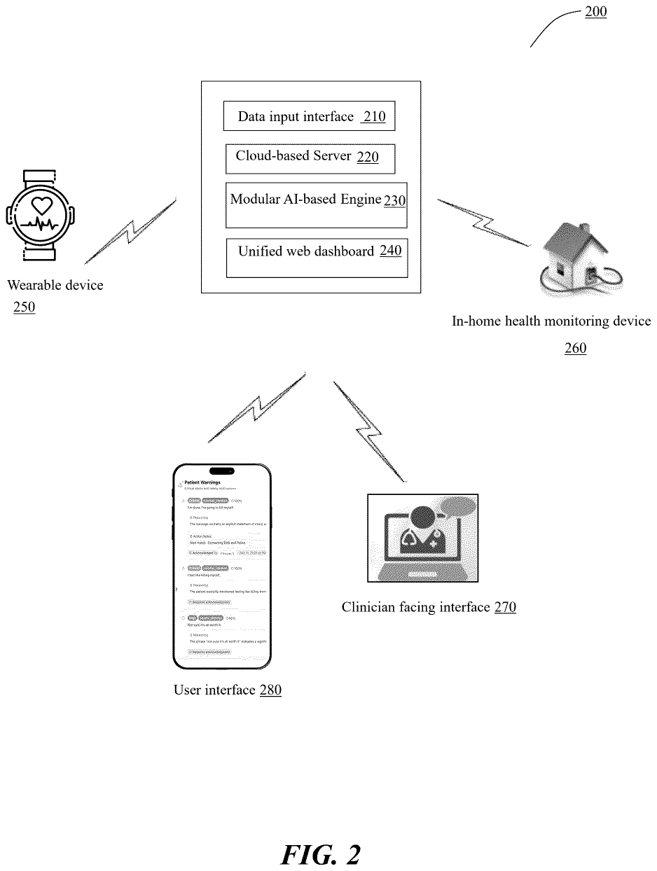

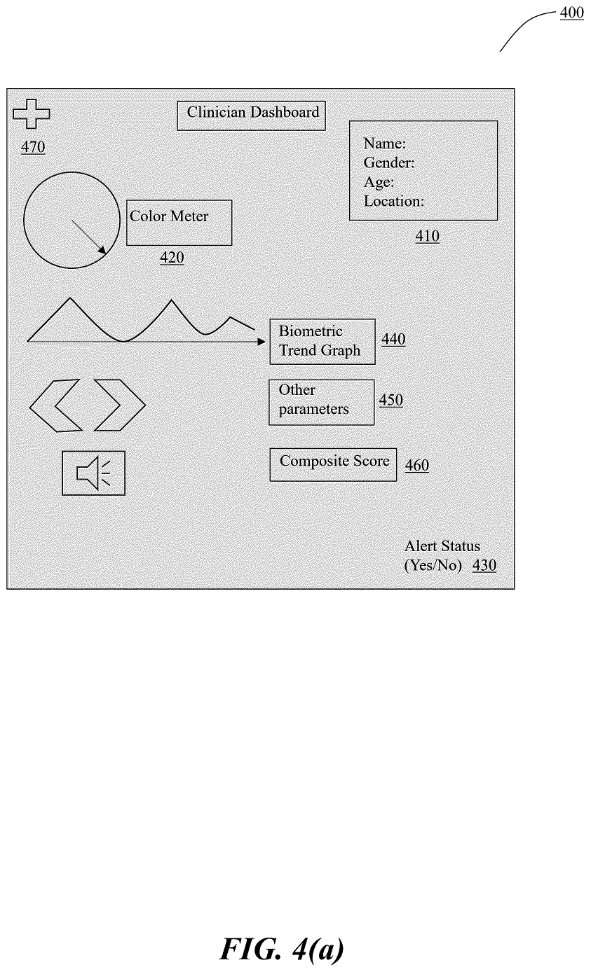

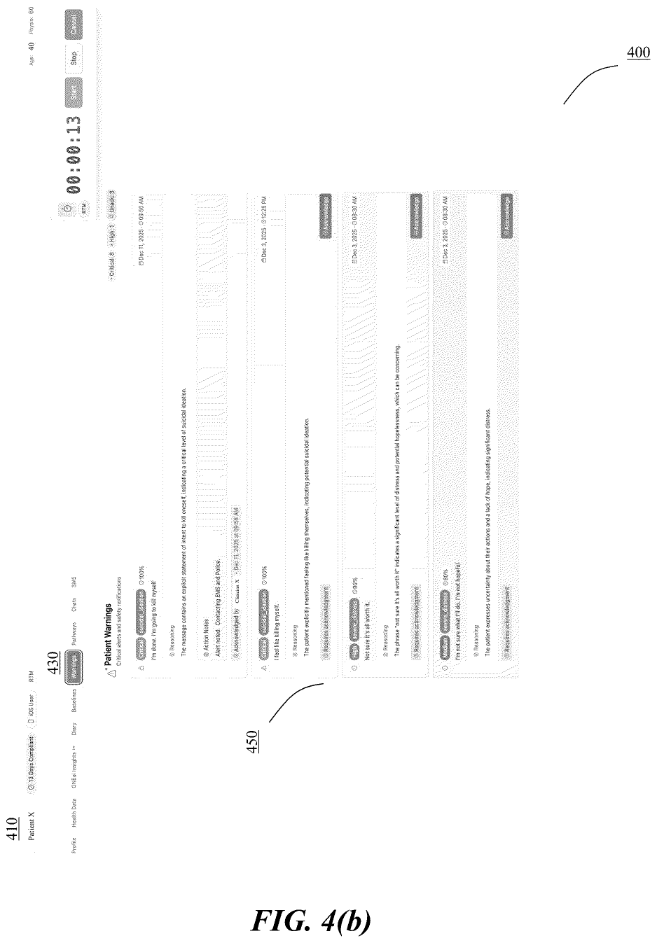

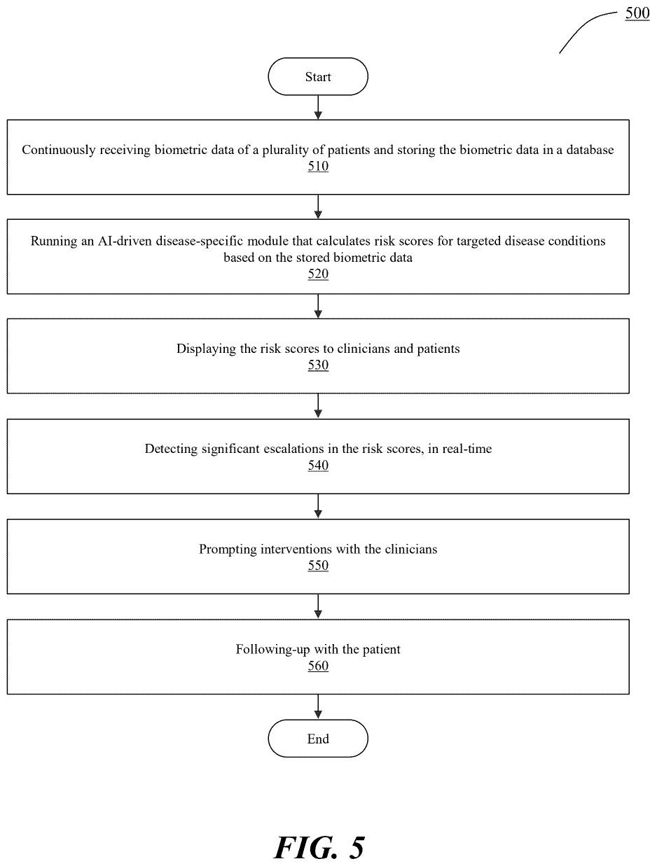

The embodiments described herein may be implemented using the system architecture illustrated in FIGS. 1 and 2 and the corresponding method operations shown in FIGS. 4 and 5. It will be appreciated that the system and method embodiments may be implemented individually or in combination.

FIG. 1 illustrates a system architecture for continuous health-related data aggregation and AI-driven health analytics, in accordance with an embodiment of the present invention. The system 100 may include a cloud-based server 110 (hereinafter referred to as ‘server’) and a dashboard 120. The server 110 may receive continuous health-related data pertaining to physiological parameters or physiological measurements in a user's body. The server may be configured to receive health-related data for a plurality of users. Further, the server 110 may store and processes the health-related data pertaining to the physiological measurements for creating individualized baselines for each user of the plurality of users.

In accordance with an embodiment of the present invention, the pre-defined period for generating the individualized baselines from the physiological measurements may vary from minimum count of days as per remote therapeutic (RTM) guidelines to a maximum limit set by a clinician. The span for monitoring a particular user is set by the clinician for getting optimized analysis.

The health-related data may be collected by a wearable device 130 or sensor-based health monitoring devices 140 (or in-home health monitoring devices). The health-related data may relate to a plurality of biometric parameters and may be one of the following: heart rate (HR), heart rate variability (HRV), blood oxygen saturation (SpO₂), steps or activity, sleep metrics, and stress scores, among others. biometric data.

The biometric parameters may be collected by the wearable device (hereinafter referred to as ‘wearables’) 130 worn by a user. The wearables 130 may be one of a fitness tracker, smartwatch, a smart bracelet, a smart belt, and a smart ring remotely connected to the cloud-based server 110 among others. In an example, the wearables 130 may include more advanced devices like, wearable blood pressure cuffs and electrocardiography (ECG) monitors which render precise tracking of cardiovascular health and other critical parameters.

The wearables 130 may include a plurality of sensors to enable such measurements in the user's body. The server 110, wearables 130, and the dashboard 120 form an interconnected health monitoring platform (or AI-powered monitoring platform).

The sensor-based health monitoring devices 140 (or in-home health monitoring devices) may include glucose monitoring devices, blood pressure measuring devices, pulse oximeters, smart thermometers, smart inhalers, connected weighing scales, environmental sensors, motion sensors, and smart pill dispensers, among others.

It is to be noted that the wearables 130 may rely on internet of things (IoT) connectivity to share the biometric data to the clinicians through the cloud-based servers 110, ensuring prompt responses or intervention in case of high-risk levels of disease conditions. For example, narrowband IoT (NB-IoT) which is a cellular technology for low-power devices communicating over long distances could be used by the wearables.

The wearables 130 and the in-home health monitoring devices 140 (combinedly referred to as ‘tracking devices’) also empower users by offering a clear view of their bio metrics, promoting better self-management. These tracking devices provide reliability, accuracy, and seamless integration into the interconnected health monitoring platforms.

In an example, the wearables 130 may include heart rate monitors, accelerometers, and gyroscopes, along with temperature sensors. Through measuring the patient's pulse, heart rate monitors provide insights into cardiovascular health. Further, the accelerometers track movement and physical activity levels, while gyroscopes help in determination of orientation and rotation, and the combination is used for motion detection. By checking the body temperature, temperature sensors contribute to overall health assessments. These sensors collectively enable the wearables 130 to analyze the biometric data effectively, providing the patients and the clinicians with valuable data for remote therapeutic management.

The sensors along with pre-stored algorithms monitor the biometric data in real-time. For instance, heart rate monitoring is achieved through photoplethysmography, which measures blood flow changes. Additionally, wearables 130 can measure blood oxygen levels using pulse oximetry technology, thus providing insights into respiratory health.

Furthermore, for sleep tracking, the accelerometers present in the wearables 130 detect any movement and patterns during sleep cycles of the patient. Additionally, wearables 130 monitor the sleep patterns by tracking various physiological metrics during sleep, like blood oxygen levels to provide insights into sleep quality and duration. The wearables 130 can accurately differentiate between sleep stages, such as light, deep, and REM sleep, thereby allowing the patients to understand their sleep cycles better.

For measuring the stress levels, stress scores or indexes may be measured by the wearable 130 to offer a proprietary stress index via HRV or electrodermal activity (EDA). In an example, a stress score above 150 can indicate excess stress. It is to be note that. A resting heart rate (RHR) counts how many times a heart beats in a minute when one is at rest, whereas the HRV measures the variation in time between each heartbeat metric. The tiny fluctuations between the heartbeat metric are measured in milliseconds and are known as R-R intervals. In an example, if a patient's heart beats 60 times in a minute, those beats may not be necessarily spaced evenly. One interval might be 980 ms, and the next may be 1030 ms. That difference between the two beats refers to as the heart rate variability or HRV. The higher the HRV, the more flexible and resilient the body of a patient is and the body can smoothly shift between stress and recovery, in contrast, a low HRV means the stress response of the patient's body is never turned off and the body stays on alert, even when one is trying to rest.

The wearable 130 may be calibrated to measure the above listed parameters so that there is an ignorable difference between lab results in clinic and that of the values shared by the wearable.

In an aspect, the value of the physiological measurements collected from the wearables 130 could be shared by an application installed in a digital device like smartphone or a tablet etc.

In another aspect, the values of the physiological measurements may directly be shared with the server 110 through the wearable 130. In such a case, an application shall be installed in the wearable 130 for establishing communication with the server.

The server 110 receives the data pertaining to the plurality of biometric parameters (shortened as ‘biometric data’) from the wearable 130 and from the in-home health monitoring device 140) and store the received data either into a single datastore categorized for storing multiple types of information or separate datastore (the datastores are not shown in the figures). The datastore may store the health-related data of the patient for retrieval and processing. In the datastore, the health-related data of the patient may be stored in variety of forms. In an example, the health-related data of the patient may be stored in a structured or unstructured or semi-structured form.

Further, to ensure data privacy and security, sensitive health-related data of the patient may be anonymized in the datastore. Encryption protocols may be employed to safeguard both the data and its representations during storage and transmission.

In an example, the health-related data of the patient could be stored as graph or hybrid of multiple forms. In another example, the health-related data of the patient may be stored in any other form or format for an efficient retrieval. Herein patient related data refers to the biometric data and targeted disease conditions. The biometric data is then processed to calculate a composite score (CS) by a composite scoring engine.

The server 110 can be accessed by the clinician via an application dashboard (referred to as a ‘unified web dashboard’ explained in below FIG. 2) on a display device for checking the CS. In an example, the clinician may be a healthcare professional.

FIG. 2 illustrates a block diagram of the system components and modules that cooperate to perform the operations of the invention, in accordance with an embodiment of the present invention.

The system 200 may include a plurality of data input interfaces 210, the cloud-based server 220, a modular AI-based engine 230, and the unified web dashboard 240. Further, the system 200 may be connected to the database(s) and wearables 1, 2, . . . n (other wearables can be connected with the system and are not shown in the FIG. 2 for the sake of brevity) 260. It is to be noted that the server 110 and 210 and the wearables 130 and 250 are equivalent in functionalities. It is also to be noted that depending upon the requirement of users or patients (if suffering from diseases-conditions) for AI-driven health analytics, any number of wearables 250 could be connected to the system 200.

The plurality of data input interfaces 210 may be interfaces of wearables 250 or the in-home health monitoring devices 260, smartphone sensors (for example, accelerometers and gyroscopes), and medical-grade equipment (for example, arm patches).

The cloud-based server or the server 220 helps in centralizing and storing the physiological measurements related data for a comprehensive analysis and accessibility across various platforms. By way of the storage of the user health-related data at the servers 220, the clinicians can access the data at anytime, anywhere, for continuous clinical surveillance better based and swift intervention. The user health-related data may be stored as individual electronic health records (EHRs) in libraries with complete information of course of treatment. The EHRs can then be analyzed using disease specific AI modules to identify trends, predict potential health risks, and personalize treatment plans. Moreover, advanced analytics tools, integrated within the server may allow for predictive modelling for chronic disease management.

The disclosed system 200 offers end-to-end encryption, role-based access control, audit trails for data review thereby complying with standard regulatory frameworks, such as the health insurance portability and accountability act (HIPAA) in the U.S. The present system 200 also supports anonymized or pseudonymized data usage for AI model training wherever mandated by regional laws considering the general data protection regulation (GDPR) in Europe. Such regulations regulate that collection, storage, and processing of the health-related data of the patients must comply with the protection of individual privacy. Moreover, the present cloud-based architecture is designed to handle large patient populations and 24/7 data ingestion.

The server 220 receives data streams from the plurality of wearables 250 or in-home health monitoring device 260 connected via a remote connection and store the individualized baselines generated from the data streams.

In an example, the wearables 250 and in-home health monitoring devices 260, equipped with the sensors, collect the biometric data and then use remote connection technologies to send the data to the server 210. The remote connection may be established primarily through a variety of wireless communication technologies.

In another example, the wearables 250 may be paired with the digital devices like smartphones or computers and then the collected data may be shared with the server 220.

In some embodiments, low-power wide-area network (LPWAN) technologies, such as LoRaWAN or Narrowband IoT (NB-IoT), may be employed to enable long-range communication with minimal power consumption. Such technologies are particularly suitable for battery-operated sensors in the wearables 260 and in-home monitoring devices deployed in environments with limited infrastructure or requiring deep indoor coverage. These communication protocols support continuous clinical surveillance by enabling periodic or event-driven transmission of health-related data over extended durations.

In certain embodiments, short-range wireless communication protocols, such as Bluetooth Low Energy (BLE), ZigBee, or Z-Wave, may be used to interconnect wearables 250, in-home health monitoring devices 260, with the server 220. For instance, BLE is commonly used for direct communication between wearables and digital devices, such as smartphones or tablets, for energy efficiency reasons thereby allowing continuous data transfer without significantly draining the wearable's battery. Due to the widespread adoption of Bluetooth in fitness trackers and smartwatches, this method of transmission is commonly validated which enables real-time monitoring and data synchronization with mobile applications. While ZigBee or Z-Wave may support mesh-based sensor networks within a residential or assisted-living environment. Such configurations allow physiological data, activity data, or environmental data to be collected from multiple devices and aggregated locally prior to transmission for continuous clinical surveillance.

In further embodiments, wireless local area network technologies, such as Wi-Fi or Wi-Fi HaLow, may be utilized to support higher data throughput and extended coverage within indoor environments. Wi-Fi HaLow, operating in sub-1 GHz frequency bands, may provide improved signal penetration through walls and reduced power consumption compared to conventional Wi-Fi, thereby supporting reliable transmission of real-time or near-real-time health-related data from the in-home health monitoring devices 260. These technologies may facilitate continuous clinical surveillance by enabling timely data transfer to the system 200 for risk analysis.

In some implementations, cellular communication technologies, including fourth-generation (4G) and fifth-generation (5G) cellular networks, may be employed to support mobility and wide-area connectivity for wearables 250 and in-home health monitoring devices 260. Cellular IoT connectivity enables data transmission during patient movement or outside a fixed residential environment, thereby supporting continuous clinical surveillance across diverse locations. In addition, location-based technologies, such as Global Positioning System (GPS), may be integrated with wearables 250 to provide mobility or activity context, which may be combined with physiological measurements to enhance assessment during continuous clinical surveillance. The described communication technologies may be used individually or in combination, and the selection of a particular technology may depend on factors such as power consumption, data rate, coverage requirements, and deployment environment.

The modular AI-based engine 230 hosts a plurality of disease-specific AI modules for analyzing different aspects of health on the collected physiological measurements using the individualized baselines. In an example, by monitoring vascular indicators (e.g., near-systolic and near-diastolic measurements) stroke risk and congestive heart failure (CHF) can be predicted and through tracking behavioral health indicators early depression and stress can be detected by the plurality of disease-specific AI modules. Similarly, by monitoring systolic blood pressure trend analysis, secondary stroke risk predictions can be done, whereas activity and sleep analysis helps in providing data-driven insights on sleep quality and daily step trends to patients, by means of the plurality of disease-specific AI modules.

In an embodiment, the modular AI-based engine 230 includes a composite scoring engine and the rules-based alert module.

In accordance with another embodiment, the composite scoring engine may refer to an AI or ML based engine to generate a composite score (CS) that reflects the individual's health risk level. The AI or ML based engine may apply smoothing/rolling averages to the historical trends or graphs so as to avoid rapid toggling between states. The AI or ML based engine may act as a dedicated composite scoring engine to parallelly process multiple types of data inputs (biometric data and other parameters related data) thereby speeding up the overall inference process.

The composite scoring engine calculates timed updates to an individual's health risk level based on the received data streams (biometric data) mapped with respect to individualized baselines (the CS may be compared with a threshold CS value and based on the comparison a risk status may be generated) and other inputs from users, for example, mood related questionnaire. The rules-based alert module issues alert to the clinicians when threshold values corresponding to the calculated health risk level of the plurality of biometric parameters may be exceeded. For example, sustained changes in the biometric parameters exceeding clinically relevant cutoffs (e.g., a 20% reduction in step count for 3+ days).

In an example, the real-time risk indicators can be displayed as one of risk scores and data trends and the personal health insights may be displayed as specific exercise suggestions.

In another example, the displayed risk indicators may be indicated by at least three-colored levels, the method may include performing one or more additional steps, namely (A) green colored level indicating a stable patient condition, (B) yellow colored level indicating a moderate risk condition of the patient, and (C) red colored level indicating a high-risk condition of the patient.

In a further example, the system 200 may also include a mobile application that provides the patient with a personalized health feedback, educational health resources, and immediate access to clinician assistance upon displaying a red colored level risk alert in real time. Real time in the present context implies during collecting the biometric data and ongoing implementation of the disease specific modules. The mobile application could easily be installed in a smartphone or any other smart device implementing the system and can be easily accessed by the patient. The mobile application could remain active in the background of a mobile phone and share biometric data obtained by the wearable device. This way the patient can easily check out his or her overall health status and seek out timely help.

In an exemplary embodiment, the mobile interface for displaying the risk status may be tracked by the patient or family member or any other relevant person in the vicinity of the patient in case off an emergency.

In another exemplary embodiment, the mobile interface for displaying the risk status may be tracked by a personal caregiver.

Further, the modular AI-based engine 230 includes at least one large language model (LLMs) configured to generate text-based clinical summaries of data trends of the user's physiological measurements. The AI-driven summarization of large data sets for quick interpretation (e.g., ChatGPT-like LLM integration) automates the process of taking clinician notes. Further, compatibility of the AI enabled health analytics platform with RTM workflows enables near real-time monitoring, intervention, and billing (if applicable), while preserving clinician oversight and decision authority. The modular AI-based engine 230 also refines threshold levels of the plurality of biometric parameters for disease conditions, baselines, and predictive models over time, leveraging population-level data or individual patient feedback to enhance accuracy and relevance.

In an example, fine-tuning and prompt-engineering techniques enable adaptation of these models to specific clinical contexts, patient populations, or disease categories without retraining entire model architectures. In addition, feedback derived from real clinical settings may be used to iteratively refine model behavior, address performance variability across specialties, and improve reliability in complex or rare disease conditions.

It is to be noted that the modular AI-based engine 230, the composite scoring engine, and the rules-based alert module may include custom algorithms, logic functions, executed by GPUs or microcontroller(s), such as ARM processors, x86 processors, DSPs or ASICs. Particularly, DSPs are designed for real-time signal processing tasks such as audio, video, and telecommunications and used in speech recognition and audio processing software. ASICs may be custom-designed for specific software tasks and thus may be used in high-efficiency computing for niche applications.

The unified web dashboard 240 includes a clinician-facing web dashboard or interface 270 and a consumer-facing interface (web or mobile) 280. The clinician-facing web interface 270 delivers real-time alerts and risk scoring (via stoplight or numeric indicators), and patient management tools. Also, the clinician-facing web interface 270 depicts in-depth historical views, day-to-day or hour-to-hour comparisons, and correlation analytics (e.g., how changes in sleep correlate with BP fluctuations). In addition, patient roster is depicted with filtering (e.g., show only those flagged as red) and integration with EHR systems is shown via standard APIs like health level seven international (HL7)'s fast healthcare interoperability resources (FHIR) which is a standard for exchanging health information electronically.

Further, through the consumer or user facing interface 280, end-users or patients can track personal health trends, receive notifications as personal health insights, and access educational resources. The user facing interface 280 may provide dynamic charts, easy-to-understand indicators, and self-care tips. Moreover, the user facing interface 280 may provide optional mental health or lifestyle questionnaires, daily check-ins, or medication reminders, alerts and nudges (e.g., prompts to increase daily steps if activity trends are declining).

It is to be notes that the unified web dashboard 240 is communicatively connected to the server 220 and can be generated upon combining analysis from the implementation of the plurality of disease-specific AI modules. Therefore, by combining the analysis, from the multiple disease-specific AI modules, the unified platform can be provided to the clinicians for comprehensive understanding of complete health of the patients.

The system 200 is also configurable through an interface to enable integration or updation of disease-specific AI modules, without disrupting existing services provided by the unified web dashboard. Therefore, the system 200 provides a modular framework for integrating future AI modules focused on additional disease conditions or sub-specialties (e.g., diabetes, chronic obstructive pulmonary disease (COPD), and post-operative care). This allows continuous expansion of the existing AI-powered monitoring platform's analytical capabilities without disrupting existing core functionalities.

Thus, as disclosed in the present invention, the AI-driven health analytics platform can automatically identify when data indicate a worsening condition, prompting interventions, telehealth consultations, or medication adjustments under recognized RTM billing frameworks.

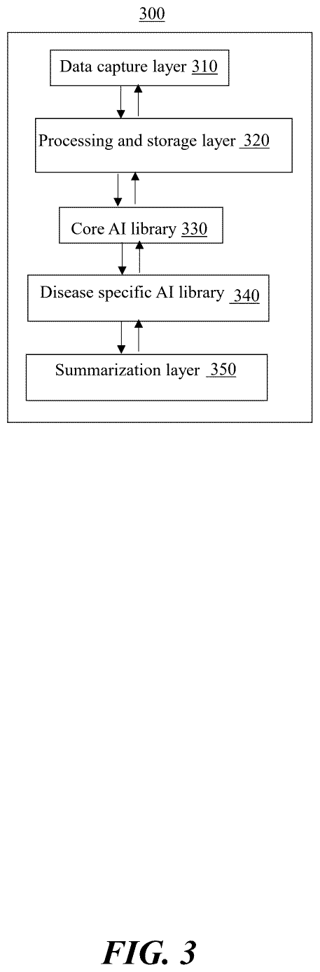

FIG. 3 illustrates an exemplary layered system architecture for continuous health-related data aggregation and AI-driven health analytics, in accordance with one or more embodiments of the present disclosure.

The system architecture 300 includes a data capture layer 310, processing & storage layer 320, a core AI library 330, a disease specific AI library 340, and a summarization layer 350.

The data capture layer 310 integrates input data from the wearables 130, 250 via APIs with wearable and in-home health monitoring devices 260 (including digit-based BP monitors, glucometers, pulse oximeters, HRV sensors, temperature probes, etc.). Also, the data capture layer 310 aids in streaming real-time or periodic biometric updates to a secure, HIPAA-compliant backend.

The processing & storage layer 320 is a cloud-based layer implying that it operates with a central database that aggregates raw biometrics, storing them alongside patient or user profiles. Also, through this layer a queue-based or event-driven mechanism triggers disease specific AI modules upon data arrival or at scheduled intervals (e.g., daily summary, multi-hour checks). The processing & storage layer 320 includes one or more processors configured to implement one or more AI-based modules for analyzing health-related data and generating risk levels.

The core AI library 330 houses baseline analytics for vitals trending (heart rate, HRV, blood pressure, SpO₂, steps, sleep). The disease specific AI library 340 includes condition-specific AI Modules. Herein following below modules can be present in the disease specific AI library 340.

CHF Monitoring & Risk Trender: This AI module evaluates systolic/diastolic equivalents from finger-based devices like pulse oximeters, smart rings, etc. and flags upward trends in near real-time.

Stroke Risk Systolic Trend: Such AI module aggregates BP values every 15 minutes, calculates daily means, and identifies significant multi-day escalations in the BP values.

Behavioral Health Indicator: This AI module merges biometrics (steps, sleep, HRV) with short mood questionnaires to detect early depressive or stress states.

Future AI Extensions: The AI-powered monitoring platform is designed to incorporate newly developed or third-party disease-specific models without service interruptions.

The summarization layer 350 employs large language models (LLMs) or advanced NLP components (ChatGPT-like) to generate short, clinically interpretable summaries of complex datasets. The summaries can highlight key trends, anomalies, or threshold breaches, thus reducing manual review time for the clinicians.

In an exemplary embodiment, the disclosed system may include an expandable AI Framework. Some examples of the new disease specific AI modules may include:

Diabetes Management Module: This module may be aimed at analyzing blood glucose logs, meal patterns, activity to prevent hyper/hypoglycemic events.

COPD Exacerbation Predictor: Such a module may be assigned for integrating respiratory rate, SpO₂, and cough frequency sensors to anticipate pulmonary decline.

Post-Operative Recovery: This module may track wound healing metrics, vital signs, and activity to identify infection or complications early.

In another exemplary embodiment, the disclosed system architecture may employ machine learning. In an example, the AI modules may be trained to learn each individual's normal baselines and adjust thresholds to minimize false alarms (e.g., naturally low HR for athletes or slightly elevated baseline BP for certain demographics). In another example, the disclosed system architecture may aggregate population-level patterns for more robust risk prediction.

In another exemplary embodiment, the disclosed system architecture may include a feedback loop. A clinician's input (validating or correcting flagged events) and responses from the users or patients (e.g., confirmation of symptoms) may be fed back into the AI-driven health analytics platform's training datasets. Over time, the system may refine its predictive accuracy, improving detection of truly significant variations in the physiological parameters.

FIG. 4(a) illustrates an exemplary view of a unified web dashboard 400 (shortened as ‘dashboard’ hereinafter) depicting the AI-driven health analytics platform (or AI-powered monitoring platform), in accordance with an embodiment of the present invention. The dashboard 400 represents a web or mobile application and is custom-built for an end user, i.e., the clinician in the present case. The dashboard 400 may be accessed by the clinician through the web browser on a computer device or he may download an application store and install on his device. The dashboard 400 may be displayed on a clinician device which may be a touchscreen device, for example, a smartphone, tablet, or other computing device.

The dashboard 400 may include a central area that occupies a display screen of the clinician device. The central area of the dashboard 400 may indicate brief patient details 410, such as name of the patient, age, gender, and location. Further, the central area is designed for displaying stoplight statuses (green, yellow, and red) 420 or alert statuses 430, short historical trend graphs 440, and the other parameters related data 450 (ex, patient responses to mood related questions). Also, the central area highlights the CS 460. The central area of the dashboard 400 integrates near real-time updates under the automated remote continuous clinical surveillance to help the clinicians rapidly identify critical changes. In addition, the displaying stoplight statuses (green, yellow, and red) 420 provides a quick visual indicator for taking action in threatening situations.

Moreover, the alert status may directly be shown with red color indicator in the dashboard 400 or an audio may be integrated for better triggering.

In an exemplary embodiment, a plus icon 470 may be displayed to the top left of the display screen. The plus icon 470 may provide plurality of options to the clinician. In an example, the plus icon 470 may enable the clinician to activate a voice-based input functionality, allowing for hands-free operation and voice commands. In another example, the plus icon 470 may include a settings tab for enabling customization options, such as language, voice command or video input, etc.

In addition, the self-report area of the display screen may display arrow signs icon for accessing previous data.

FIG. 4(b) illustrates another exemplary view of a unified web dashboard 460 depicting AI-driven health analytics with automated remote continuous clinical surveillance, in accordance with an embodiment of the present invention. In FIG. 3(b), the elements common to FIG. 4(a) retain the same reference numerals 410, 430, and 450. The integrated clinician dashboard 460 displays exemplary potential warnings and other parameters like patient responses to behavioral questions along with colored patterns. The integrated clinician dashboard 460 also captures the date and time information of the instances when the questions have been addressed by the user. Also, acknowledgement of the critical warning from the clinician is displayed along with the action notes and reasoning for future references. The action notes list the action taken based on the reasoning for the swift intervention by the clinician.

FIG. 5 illustrates a flowchart of a method 500 for performing continuous clinical surveillance using disease-specific AI modules, in accordance with an embodiment of the present invention. The method begins with step 510 which includes continuously receiving biometric data of a plurality of patients and storing the biometric data in a database.

At 520, the method may include running an AI-driven disease-specific module that calculates risk scores for targeted disease conditions based on the stored biometric data.

At 530, the method may include displaying the risk scores to the clinicians and a patient of the plurality of the patients.

At 540, the method may include detecting significant escalations in the risk scores in real-time.

At 550, the method may include prompting interventions with the clinicians, upon the above detection of significant escalations in the risk scores in real-time.

At 560, the method may include following-up with the patient, upon the above detection of significant escalations in the risk scores in real-time, and the method may include performing one or more additional steps. Here, the significant escalations in the risk scores may correspond to either consistent or sudden rise in values of the one or more biometric parameters pertaining to the biometric data, beyond respective threshold values of the biometric parameters. Also, the changes may be indicative of an elevated risk of an adverse physiological condition, like CHF decompensation or an imminent stroke, thereby triggering an alert to the clinician for urgent clinical evaluation

In an exemplary embodiment, the risk scores for targeted disease conditions may be calculated by a composite scoring engine based on individualized baselines for the targeted disease condition and trending patterns of the biometric data as has been explained above for FIG. 2. Each parameter is stored and analyzed for at least 7-14 days to create a personalized baseline. Then these baselines are used for ongoing comparisons under the continuous clinical surveillance method.

In an exemplary embodiment, the risk scores may be indicated at least in a color-coded, text-based format, and graph-based format. By way of presentation of critical health related information in color and picture formats different ways, the clinician may easily differentiate from a normal situation to emergency situation and move on to the next patient if one case is resolved just by checking the risk status through the risk scores.

In certain embodiments, the targeted conditions may be one stroke risk, depression, and congestive heart failure (CHF), among others.

In an exemplary implementation, clinician or patient-confirmed outcomes may refine the AI models'predictive capabilities over time through a feedback loop.

In another exemplary implementation, the clinicians and patients may be authorized and the authorization may include an agreement to remote therapeutic monitoring (RTM) guidelines.

In an exemplary embodiment, the interventions may include sending an alert or a short message from a telehealth consultant to one of a family member or a personal caregiver or a relevant healthcare professional or emergency services. By timely tracking the risk status intervention can be done to prevent severity.

In case of senior citizens or persons with disabilities, the personal caregiver may be responsible for complete supervision of the health of the patient. Thus, the personal caregiver may check the mobile interface for timely intervention.

In an exemplary embodiment, the follow-up may include an in-person appointment with the clinician or change in type or schedule of medications of the patient based on the risk level.

In an exemplary embodiment, the biometric data may be fetched from a plurality of wearable devices. The wearable device may include one of a smartwatch, a smart bracelet, a smart belt, an earbud, a smart ring, and a smart clothing remotely connected to the database. the database is similar to the datastore discussed in the above FIG. 2.

Post-deployment, the method 400 may be optimized with mechanisms for continuous learning. Patient data may be anonymized and analyzed to identify improvement areas, and periodic updates may be applied to refine the capabilities of disclosed system.

Throughout the implantation of the continuous clinical surveillance process, adherence to the RTM guidelines is maintained.

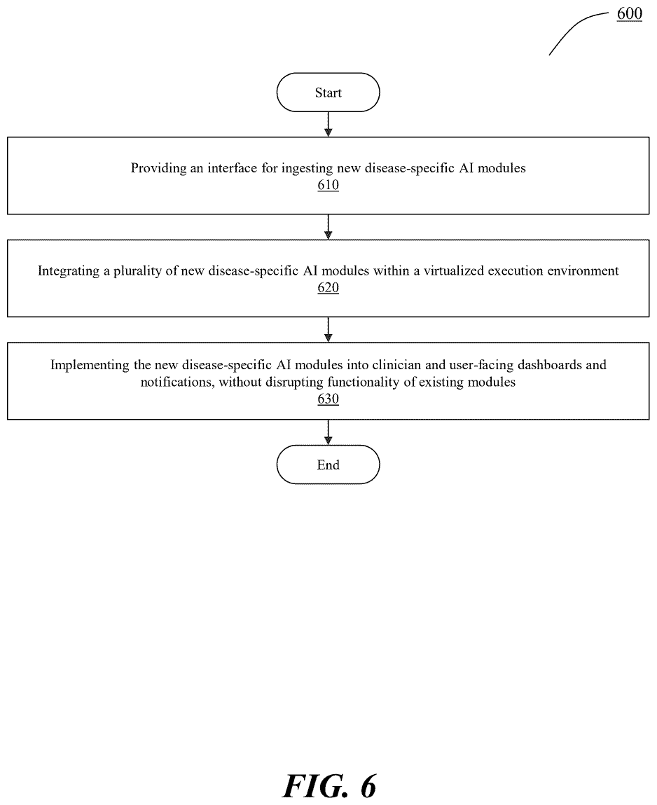

FIG. 6 illustrates of a method of deploying a new disease-specific AI module in an existing AI-powered monitoring platform, in accordance with an embodiment of the present invention.

At 610, the method may include providing an interface for ingesting new disease-specific AI modules. Herein, ingestion refers to the process of importing, transferring, or loading data pertaining to disease-specific AI modules from various external sources into the system or storage infrastructure like the database where it can be stored, processed, and analyzed.

At 620, the method may include integrating a plurality of new disease-specific AI modules within a virtualized execution environment.

At 630, the method may include implementing the new disease-specific AI modules into clinician and user-facing dashboards and notifications, without disrupting functionality of existing modules. Herein, it is to be noted that the interface may be part of a unified web dashboard, generated upon execution of a plurality of disease-specific algorithms in the virtualized execution environment.

A virtualized execution environment may refer to a logically isolated runtime environment where any algorithm or instructions or module is executed independently of other software entities, while sharing same computing resources, such as, operating systems (OS) and hardware. Such environments may be dynamically instantiated, modified, or terminated, without requiring hardware or OS modifications.

In an exemplary embodiment, a virtualized execution environment may be implemented using operating-system-level virtualization, hardware-assisted virtualization, or application-level isolation techniques. For instance, the virtualized execution environment may comprise container-based execution instances, virtual machines, sandboxed runtime processes, or isolated software instances managed by an orchestration layer. These examples are illustrative and non-limiting, and other mechanisms for providing virtualized execution contexts may be employed without departing from the scope of the disclosure.

In a preferred embodiment, the plurality of disease-specific AI modules and the new disease-specific AI modules may be integrated in a containerized environment.

In an exemplary embodiment, a version-control system of the disease-specific AI modules ensure backward compatibility and stable rollout to the clinicians and the users.

A containerized architecture describes encapsulating an application and its dependencies into a portable, lightweight container which could be easily deployable in a variety of computing environments. Containers are the central component of the containerization architecture. These containers use virtualization at operating system level to guarantee consistent runtime environments independent of the supporting infrastructure. The containers are instances of isolated environments that contain all the necessary code, runtime, system tools, libraries, and settings to run an application.

Containerization further enhances system portability and consistency by abstracting application dependencies from underlying infrastructure of the system. Because containers include all necessary runtime components, they can be deployed across heterogeneous environments with minimal configuration. This improves reliability and reduces deployment errors. In distributed architectures, containerization facilitates modular development and independent deployment of individual services, enabling updation, scaling, or replacement of discrete components of the overall system without impacting other components.

In addition, containerization integrates readily with continuous integration and continuous deployment (CI/CD) pipelines, enabling automated building, testing, and deployment of application components of the disease specific AI modules. By using standardized container images and orchestration frameworks, such as Kubernetes, containerized systems build by disease specific AI modules can dynamically scale in response to heavy health-related data of the patients. Additionally, the containerized systems perform health monitoring along with load balancing thereby improving application resilience and operational efficiency in distributed environment of AI-driven health analytics.

In an exemplary embodiment, the interface may be a standardized application programming interface (API).

In an exemplary embodiment, the existing modules may include a plurality of disease-specific algorithms relating to diseases from one of congestive heart failure (CHF) risk, stroke risk, and behavioral health, etc.

The disclosed method is capable of being implemented with multiple wearable devices which could be worn by the patient and are also connected to the server for biometric data monitoring.

The unified web dashboard or other interfaces (combinedly referred to as ‘interface’) which have been discussed in the present invention may be designed using the coding languages such as, javascript, python, and SQL, etc. Further, various open-source frameworks such as Note JS, flutter, react native, Xamarin, iconic framework, codova, asp.net, and nativescript, etc. may be employed to create the frontend. The disclosed interface support multimodal interaction by enabling users to toggle between various input methods seamlessly. Additionally, the interface adapts to different screen sizes and resolutions, thereby ensuring optimal rendering of the output across various devices used by the patients and the clinicians.

In some embodiments, the disclosed systems and methods may be applied to rehabilitation and recovery monitoring following surgery or injury. Wearables and in-home health monitoring devices may collect health-related data indicative of patient recovery, including physical activity levels, movement patterns, muscle usage, and mobility metrics. Motion sensors, accelerometers, and related sensing components may be used to generate objective measurements that support assessment of rehabilitation progress. The collected data may be analyzed as part of continuous clinical surveillance to evaluate the effectiveness of ongoing treatment plans and to inform adjustments for therapeutic interventions. In certain implementations, such monitoring may reduce the frequency of in-person clinical visits while enabling recovery to occur in the home environment.

In some embodiments, the disclosed systems and methods may be applied to mental health monitoring as part of continuous clinical surveillance. Health-related data associated with psychological well-being may include physiological indicators such as heart rate variability (HRV), sleep patterns, activity levels, and stress-related responses. The wearables and in-home monitoring devices may collect such data over a specific period of time, enabling longitudinal analysis of trends associated with stress, anxiety, or sleep disorders. The analyzed data may support identification of changes in mental health status and facilitate timely clinical intervention.

In some embodiments, the disclosed systems and methods may be applied to elderly care and assisted living scenarios. The wearables and in-home monitoring devices may include motion sensors and accelerometers configured to detect falls or abnormal movement patterns and generate alerts as part of continuous clinical surveillance. In certain implementations, location-related data obtained from positioning technologies may be used to monitor mobility or wandering behavior associated with cognitive impairments. Additionally, physiological measurements, such as heart rate or activity anomalies, may be monitored to provide early indication of potential health events. Such applications may support independent living while enabling timely caregiver or clinical response.

Therefore, the disclosed invention presents the unified platform as the unified web dashboard which simplifies health care management by combining multiple disease-specific AI modules, each curated within one interface.

This projects that future AI or specialized analytics can be plugged in without major platform overhauls.

With the use of wearables and the disclosed implementation using the unified web dashboard, both professional healthcare workflows (EHR integration, real-time alerts) and personal wellness tracking are packed in one system. This facilitates scaling and serviceability with minimal retraining, efforts, and cost.

Additionally, the automated summaries, near real-time alerts, and integrated data streams reduce manual workload for the clinicians while empowering patients to stay informed about their health.

In essence, the disclosed invention revolutionizes the landscape of disease monitoring and patient care by offering a scalable and resource-efficient solution.

When discussing the invention, it is important to recognize that various techniques and steps are disclosed, each offering distinct advantages and capable of being employed independently or in combination with one another. Therefore, this description avoids redundant enumeration of all possible combinations of individual steps to maintain clarity. However, it should be noted that such combinations are fully encompassed within the scope of the invention and the accompanying claims. Consequently, the specification and claims should be interpreted with the understanding that these combinations are permissible and fall within the ambit of the invention.

In a case that no conflict occurs, the embodiments in the present disclosure and the features in the embodiments may be mutually combined. The foregoing descriptions are merely specific implementations of the present disclosure, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure shall be subject to the protection scope of the claims.

The foregoing descriptions of specific embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated.

The invention claimed is:

1. A system for continuous health-related data aggregation and AI-driven health analytics, the system comprising:
a plurality of data input interfaces configured to collect a plurality of physiological measurements for a plurality of users;
a cloud-based server configured to store and process the plurality of physiological measurements to create individualized baselines for each user of the plurality of users;
a modular AI-based engine configured to implement a plurality of disease-specific AI modules on the collected physiological measurements using the individualized baselines; and
a unified web dashboard, generated upon implementation of the plurality of disease-specific AI modules, comprising:
a clinician facing interface to provide real-time risk indicators for clinicians; and
a consumer interface to provide personal health insights to the plurality of users.

2. The system of claim 1, wherein the physiological measurements are collected from one of wearable devices and sensor-based health monitoring devices.

3. The system of claim 1, wherein the physiological measurements are collected from a sensor-based health monitoring device comprising one or more sensors configured to collect at least one physiological measurement of the user in a home environment.

4. The system of claim 1, wherein the individualized baselines are generated by storing and processing the physiological measurements for a pre-defined time period.

5. The system of claim 1, wherein the disease-specific AI modules relate to diseases from one of congestive heart failure (CHF) risk, stroke risk, and behavioral health, etc.

6. The system of claim 1, wherein the modular AI-based engine comprises at least one large language model (LLMs) configured to generate text-based clinical summaries of data trends of the user's physiological measurements.

7. The system of claim 1, wherein the unified web dashboard is communicatively connected to the cloud-based server and is generated upon combining analysis from the implementation of the plurality of disease-specific AI modules.

8. The system of claim 1, wherein the system comprises an interface to enable integration or updation of disease-specific AI modules, without disrupting existing services provided by the unified web dashboard.

9. The system of claim 1, wherein the real-time risk indicators are displayed as one of risk scores and data trends and the personal health insights are displayed as specific exercise suggestions.

10. A method for performing continuous clinical surveillance using disease-specific AI modules, the method comprising:
continuously receiving biometric data of a plurality of patients and storing the biometric data in a database;
running an AI-driven disease-specific module that calculates risk scores for targeted disease conditions based on the stored biometric data;
displaying the risk scores to clinicians and a patient of the plurality of the patients; and
upon detecting significant escalations in the risk scores, in real-time:
prompting interventions with the clinicians, or
following-up with the patient.

11. The method of claim 10, wherein the risk scores for targeted disease conditions are calculated by a composite scoring engine based on threshold value detection of an individualized baseline of the targeted disease condition and trending patterns of the biometric data.

12. The method of claim 10, wherein the risk scores are indicated at least in a color-coded, text-based format, and graph-based format.

13. The method of claim 10, wherein the targeted conditions are one of stroke risk, depression, and congestive heart failure (CHF), and the like.

14. The method of claim 10, wherein the clinicians and patients are authorized by an agreement, to remote therapeutic monitoring (RTM) guidelines.

15. The method of claim 10, wherein the interventions may comprise sending an alert or a short message from a telehealth consultant to one of:
a family member or a relevant healthcare professional, and
emergency services.

16. The method of claim 10, wherein the follow-up may comprise an in-person appointment with the clinician or change in type or schedule of medications of the patient based on the risk level.

17. The method of claim 10, wherein the significant escalations in the risk scores correspond to threshold-exceeding changes in one or more biometric parameters pertaining to the biometric data, and wherein the changes are indicative of an elevated risk of an adverse physiological condition thereby triggering an alert to the clinician for urgent clinical evaluation.

18. A method of deploying a new disease-specific AI module in an existing AI-powered monitoring platform, the method comprising:
providing an interface for ingesting new disease-specific AI modules,
wherein the interface is part of a unified web dashboard, generated upon execution of a plurality of disease-specific AI modules in a virtualized execution environment;
integrating a plurality of new disease-specific AI modules within the virtualized execution environment; and
implementing the new disease-specific AI modules into clinician and user-facing dashboards and notifications, without disrupting functionality of existing modules.

19. The method of claim 19, wherein the plurality of disease-specific AI modules and the new disease-specific AI modules are integrated in a containerized environment.

20. The method of claim 19, wherein the existing modules comprise a plurality of disease-specific AI modules relating to diseases from one of congestive heart failure (CHF) risk, stroke risk, and behavioral health, etc.
